(19) United States
(12) Patent Application Publication
(10) Pub. No.: US 2024/0029851 A1
(43) Pub. Date: Jan. 25, 2024

(54) REMOTE VERIFICATION SYSTEM FOR PHARMACEUTICAL COUNTING AND PACKAGING DEVICE

(71) Applicant: RXSAFE LLC, Vista, CA (US)

(72) Inventor: William K. Holmes, San Diego, CA (US)

(21) Appl. No.: 18/029,431

(22) PCT Filed: Oct. 14, 2021
(86) PCT No.: PCT/US2021/055025

§ 371 Date: Mar. 30, 2023

Related U.S. Application Data
(60) Provisional application No. 63/091,856, filed on Oct. 14, 2020.

(51) Int. Cl. G16H 20/10; G16H 40/67

(52) U.S. Cl. CPC G16H 20/10; G16H 40/67

(57) ABSTRACT

One embodiment provides a method for remote verification for a pharmaceutical counting and packaging device (150). The method includes filling, using the pharmaceutical counting and packaging device (150), a prescription and capturing, using the pharmaceutical counting and packaging device (150), images during filling of the prescription. The method also includes transmitting, using the pharmaceutical counting and packaging device (150), captured images to a remote pharmacist device (180).

### CROSS-REFERENCE TO RELATED APPLICATIONS

This application claims priority to U.S. Provisional Patent Application No. 63/091,856, filed on Oct. 14, 2020, the entire contents of which are incorporated by reference herein.

### FIELD OF THE INVENTION

The present invention relates to remote verification systems for pharmaceutical packaging machines and, more particularly, a remote verification system for a pharmaceutical counting machine for packaging pharmaceuticals.

### SUMMARY

One embodiment provides a method for remote verification for a pharmaceutical counting and packaging device. The method includes filling, using a pharmaceutical counting and packaging device, a prescription and capturing, using the pharmaceutical counting and packaging device, images during filling of the prescription. The method also includes transmitting, using the pharmaceutical counting and packaging device, captured images to a remote pharmacist device.

Another embodiment provides a method for remote verification for a pharmaceutical counting and packaging device. The method includes receiving a prescription to be filled by the pharmaceutical counting and packaging device and scanning, using a scanner of the pharmaceutical counting and packaging machine, a bulk storage container to identify a first type of medication in the bulk storage container. The method also includes capturing, using a camera system of the pharmaceutical counting and packaging machine, an image of the bulk storage container and capturing, using the camera system, an image of a label applied to a package receiving the first type of medication. The method further includes counting, using an electronic processor of the pharmaceutical counting and packaging device, a number of medications on a counting tray of the pharmaceutical counting and packaging device and determining, using the electronic processor, whether the number of medications on the counting tray matches the number of medications needed to fill the prescription. In response to the number of medications on the counting tray matching the number of medications needed to fill the prescription, the method includes capturing, using the camera system, an image of medications on the counting tray, and capturing, using the camera system, an image of medications in the package. The method also includes transmitting, using a transceiver of the pharmaceutical counting and packaging device, the image of the bulk storage container, the image of the label applied to the package, the image of medications on the counting tray, and the image of medications in the package to a remote device.

In some aspects, the method also includes determining whether a second type of medication on the counting tray correspond to one or both of the first type of medication and a third type of medication listed on the prescription. The method further includes transmitting a first indication to the remote device when the second type of medication on the counting tray correspond to one or both of the first type of medication and the third type of medication, and transmitting a second indication to the remote device when the second type of medication on the counting tray does not correspond to one or both of the first type of medication and the third type of medication.

In some aspects, the method also transmitting the prescription to the remote device.

In some aspects, the method also receiving verification information corresponding to the prescription, wherein the verification information includes an indication of whether the package is correctly packaged by the pharmaceutical counting and packaging device.

Another embodiment provides a pharmaceutical counting and packaging device including a counting tray configured to receive medications for counting, a camera system for capturing images, a transceiver, and an electronic processor coupled to the camera system and the transceiver. The electronic processor is configured to receive a prescription to be filled by the pharmaceutical counting and packaging device, and capture, using the camera system, an image of a label applied to a package receiving a first type of medication listed on the prescription. The electronic processor is also configured to count a number of medications on the counting tray, and determine whether the number of medications on the counting tray matches the number of medications needed to fill the prescription. In response to the number of medications on the counting tray matching the number of medications needed to fill the prescription, the electronic processor is configured to capture, using the camera system, an image of medications on the counting tray, and capture, using the camera system, an image of medications in the package. The electronic processor is also configured to transmit, using the transceiver, the image of the label applied to the package, the image of medications on the counting tray, and the image of medications in the package to a remote device.

In some aspects, the electronic processor is also configured to determine whether a second type of medication on the counting tray correspond to one or both of the first type of medication and a third type of medication listed on the prescription. The electronic processor is further configured to transmit a first indication to the remote device when the second type of medication on the counting tray correspond to one or both of the first type of medication and the third type of medication, and transmit a second indication to the remote device when the second type of medication on the counting tray does not correspond to one or both of the first type of medication and the third type of medication.

In some aspects, the electronic processor is also configured to transmit the prescription to the remote device.

In some aspects, the electronic processor is also configured to receive verification information corresponding to the prescription, wherein the verification information includes an indication of whether the package is correctly packaged by the pharmaceutical counting and packaging device.

In some aspects, the electronic processor is also configured to scan, using a scanner, a bulk storage container to identify a second type of medication in the bulk storage container. The medications from the bulk storage container are received on the counting tray when the first type of medication matches the second type of medication. The electronic processor is also configured to capture, using the camera system, an image of the bulk storage container and transmit the image of the bulk storage container to the remote device.

Another embodiment provides a method for remote verification for a pharmaceutical counting and packaging device. The method includes receiving a prescription to be filled by the pharmaceutical counting and packaging device and capturing, using a camera system of the pharmaceutical counting and packaging device, an image of a label applied to a package receiving a first type of medication listed on the prescription. The method also includes counting, using an electronic processor of the pharmaceutical counting and packaging device, a number of medications on a counting tray of the pharmaceutical counting and packaging device and determining, using the electronic processor, whether the number of medications on the counting tray matches the number of medications needed to fill the prescription. In response to the number of medications on the counting tray matching the number of medications needed to fill the prescription, the method also includes capturing, using the camera system, an image of medications on the counting tray, and capturing, using the camera system, an image of medications in the package. The method also includes transmitting, using a transceiver of the pharmaceutical counting and packaging device, the image of the label applied to the package, the image of medications on the counting tray, and the image of medications in the package to a remote device.

In some aspects, the method also includes determining whether a second type of medication on the counting tray correspond to one or both of the first type of medication and a third type of medication listed on the prescription. The method further includes transmitting a first indication to the remote device when the second type of medication on the counting tray correspond to one or both of the first type of medication and the third type of medication, and transmitting a second indication to the remote device when the second type of medication on the counting tray does not correspond to one or both of the first type of medication and the third type of medication.

In some aspects, the method also includes transmitting the prescription to the remote device.

In some aspects, the method also includes receiving verification information corresponding to the prescription, wherein the verification information includes an indication of whether the package is correctly packaged by the pharmaceutical counting and packaging device.

In some aspects, the method also includes scanning, using a scanner, a bulk storage container to identify a second type of medication in the bulk storage container. The medications from the bulk storage container are received on the counting tray when the first type of medication matches the second type of medication. The method further includes capturing, using the camera system, an image of the bulk storage container, and transmitting the image of the bulk storage container to the remote device.

Other aspects of the invention will become apparent by consideration of the detailed description and accompanying drawings.

### BRIEF DESCRIPTION OF THE DRAWINGS

FIG. 1 is a schematic of a pharmacy management system in accordance with some embodiments.

FIG. 2 is a schematic of a remote pharmacist device communicating with the pharmacy management system of FIG. 1 in accordance with some embodiments.

FIG. 3 is a perspective view of a pharmaceutical counting device of the pharmacy management system of FIG. 1 in accordance with some embodiments.

FIG. 4 is a perspective view of a packaging slot of the pharmaceutical counting and packaging device of FIG. 3 in accordance with some embodiments

FIG. 5 is a schematic of the pharmaceutical counting and packaging device of FIG. 3 in accordance with some embodiments.

FIG. 6 is a flowchart of a method for filling a prescription using the pharmaceutical counting and packaging device of FIG. 3 in accordance with some embodiments.

FIG. 7 is a flowchart of a method for remote verification for the pharmaceutical counting and packaging device of FIG. 3 in accordance with some embodiments.

FIG. 8 is a flowchart of a method for remote verification in accordance with some embodiments.

FIGS. 9A-9D illustrate verification images used for remote verification in accordance with some embodiments.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way but may also be configured in ways that are not listed.

Automatic packaging of pharmaceuticals for filling prescription results in lower error rate and time saving for pharmacies. Bigger pharmacies that have multiple locations typically use automatic packaging to increase efficiency. However, the multiple locations may have different volume of pharmaceutical transactions. Typically, one or more technicians operates and handles prescriptions received at the pharmacy, while a pharmacist reviews the filled prescription for accuracy and provides instructions to the customer. In some instances, a first pharmacist at a first location may be too busy to verify all the prescriptions, while a second pharmacist at a second location may have some free time to verify the prescriptions from the first location. However, the current verification systems do not allow for distribution of work between different pharmacy locations. Accordingly, there is a need for remote verification systems for pharmaceutical counting and packaging machines.

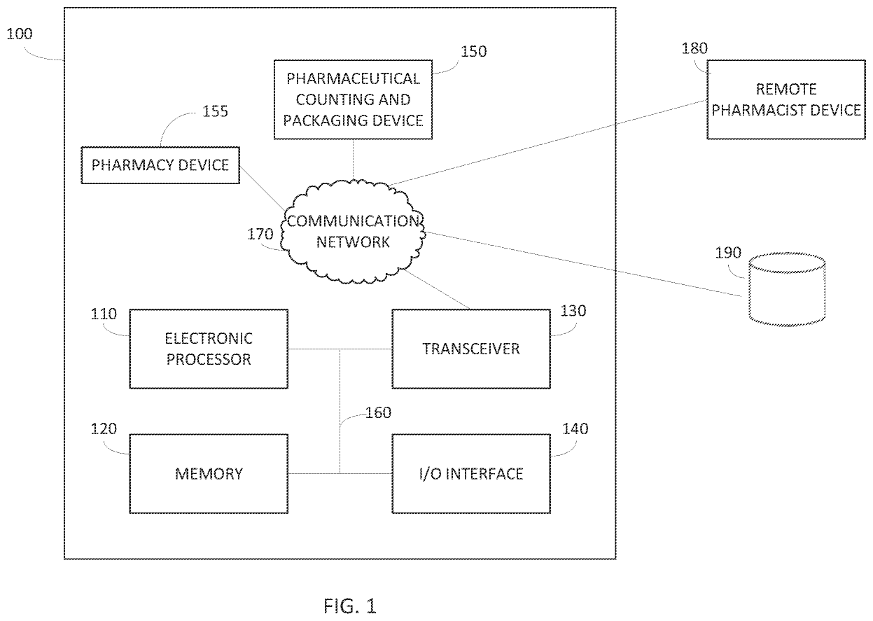

FIG. 1 is a schematic illustration of a pharmacy management system 100 according to one example embodiment. The pharmacy management system 100 is a pharmacy automation and remote verification system for use in retail pharmacies. In the example illustrated in FIG. 1, the pharmacy management system 100 includes an electronic processor 110, a memory 120, a transceiver 130, an input/output interface 140, a pharmaceutical counting and packaging device 150, and a pharmacy device 155. The electronic processor 110, the memory 120, the transceiver 130, and the input/output interface 140 communicate over one or more control and/or data buses (e.g., a communication bus 160). FIG. 1 illustrates only one exemplary embodiment of the pharmacy management system 100. The pharmacy management system 100 may include more or fewer components and may perform functions other than those explicitly described herein. For example, in some embodiments, the pharmacy management system 100 may include a pharmaceutical storage and retrieval system, an automatic strip packaging system, and/or an automatic blister card packaging system.

In some embodiments, the electronic processor 110 is implemented as a microprocessor with separate memory, such as the memory 120. In other embodiments, the electronic processor 110 may be implemented as a microcontroller (with memory 120 on the same chip). In other embodiments, the electronic processor 110 may be implemented using multiple processors. In addition, the electronic processor 110 may be implemented partially or entirely as, for example, a field-programmable gate array (FPGA), an application specific integrated circuit (ASIC), and the like, and the memory 120 may not be needed or be modified accordingly. In the example illustrated, the memory 120 includes non-transitory, computer-readable memory that stores instructions that are received and executed by the electronic processor 110 to carry out functionality of the pharmacy management system 100 described herein. The memory 120 may include, for example, a program storage area and a data storage area. The program storage area and the data storage area may include combinations of different types of memory, such as read-only memory and random-access memory.

The transceiver 130 enables wired or wireless communication from the pharmacy management system 100 to a communication network 170. In some embodiments, the transceiver 130 may include separate transmitting and receiving components, for example, a transmitter and a receiver. The pharmacy management system 100, through the communication network 170, may communicate with the pharmaceutical counting and packaging device 150, the pharmacy device 155, a remote pharmacist device 180 and databases, for example, a pharmacy database 190. In some embodiments, the pharmacy management system 100 is part of the pharmacy device 155.

The input/output interface 140 may include one or more input mechanisms (e.g., a touch screen, a keypad, a button, a knob, and the like), one or more output mechanisms (e.g., a display, a printer, a speaker, and the like), or a combination thereof. The input/output interface 140 receives input from the input devices actuated by a user, and provides output to the output devices with which a user interacts. In some embodiments, as an alternative or in addition to managing inputs and outputs through the device input/output interface 240, the pharmacy management system 100 may receive user inputs, provide user outputs, or both by communicating with an external device, such as a console computer or a remote pharmacist device 180 at the current pharmacy location, over a wired or wireless connection.

The communication network 170 can be built according to any suitable networking technology or topology or combinations of technologies and topologies and can include multiple sub-networks. Connections between the devices and systems shown in FIG. 1 can be made through local area networks (“LANs”), wide area networks (“WANs”), public switched telephone networks (“PSTNs”), wireless networks, Intranets, the Internet, or any other suitable networks. In a hospital or medical care facility, for example, communication between the devices and systems shown in FIG. 1 can be made through any required communication protocol(s), including, for example, the Health Level Seven (“HL7”) protocol or any other version of a required protocol. The HL7 protocol is a standard protocol which specifies the criteria for data exchange (including the required interface implementation) between two computer applications (sender and receiver), such that a universal standard is used by vendors, thereby facilitating the exchange of electronic data in health care environments. The HL7 protocol allows health care institutions to exchange key sets of data from different application systems. Specifically, the HL7 protocol can define the data to be exchanged, the timing of the interchange, and the communication of errors to the application. The formats are generally generic in nature and can be configured to meet the needs of the applications involved.

The pharmacy database 190 is a database implemented by the pharmacy including multiple locations for coordination of orders and management of pharmaceuticals across the multiple locations. The pharmacy database 190 can be accessed and updated by a pharmacy management system 100 at any location or by a remote pharmacist device 180 of any pharmacist of the retail pharmacy. The pharmacy database 190 stores information regarding the pharmaceuticals stocked and sold by the retail pharmacy including, for example, codes, description, images, and the like of the pharmaceuticals. The pharmacy database 190 also stores information regarding order received at each pharmacy location, for example, customer name, customer prescription, customer drug interaction, and the like.

The remote pharmacist device 180 is, for example, a computing device of a pharmacist location at another location of the retail pharmacy. In some embodiments, the pharmacist may also be a dedicated remote pharmacist who may be used for handling excess customer orders at pharmacy locations. FIG. 1 illustrates only one remote pharmacist device 180. However, the pharmacy management system 100 may communicate with multiple remote pharmacist devices 180. The pharmacy device 155 is, for example a local computing device of the pharmacy location that includes the pharmaceutical counting and packaging device 150. For example, the pharmacy device 155 is similar to the remote pharmacist device 180, but co-located with the pharmaceutical counting and packaging device 150. In some embodiments, the pharmacy device 155 is a point of sale device that is used to process medication sales to customers.

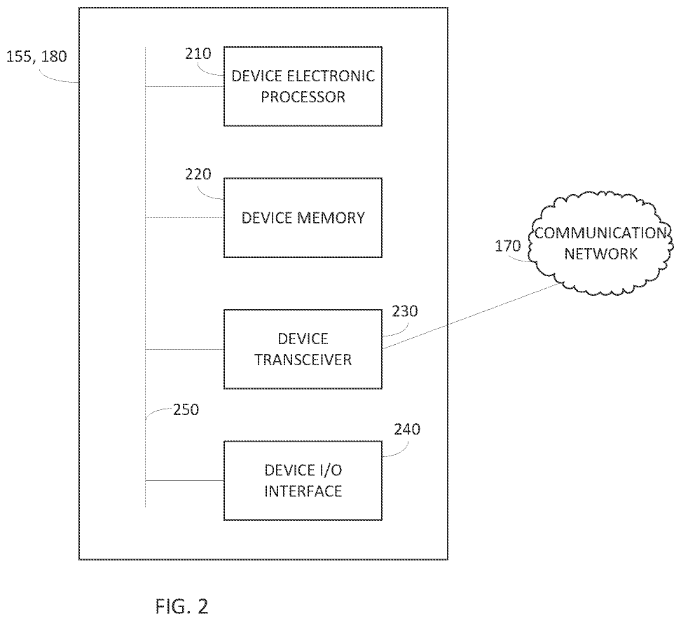

FIG. 2 schematically illustrates the pharmacy device 155 and the remote pharmacist device 180, referred to as singularly as the pharmacy device 155, 180. In the example illustrated, the pharmacy device 155, 180 includes a device electronic processor 210, a device memory 220, a device transceiver 230, and a device input/output interface 240. The device electronic processor 210, the device memory 220, the device transceiver 230, and the device input/output interface 240 communicate over one or more control and/or data buses (e.g., a communication bus 250). FIG. 2 illustrates only one exemplary embodiment of the pharmacy device 155, 180. The pharmacy device 155, 180 may include more or fewer components and may perform functions other than those explicitly described herein.

The device electronic processor 210 and the device memory 220 may be implemented similar to the electronic processor 110 and the memory 120 as described above. The device transceiver 230 enables communication from the pharmacy device 155, 180 to the communication network 170. In some embodiments, the device transceiver 230 may include separate transmitting and receiving components, for example, a transmitter and a receiver. The pharmacy device 155, 180 through the communication network 170 may communicate with the pharmacy management system 100, the pharmaceutical counting and packaging device 150 and databases, for example, the pharmacy database 190.

The device input/output interface 240 may include one or more input mechanisms (e.g., a touch screen, a keypad, a button, a knob, and the like), one or more output mechanisms (e.g., a display, a printer, a speaker, and the like), or a combination thereof. The device input/output interface 240 receives input from the input devices actuated by a user, and provides output to the output devices with which a user interacts.

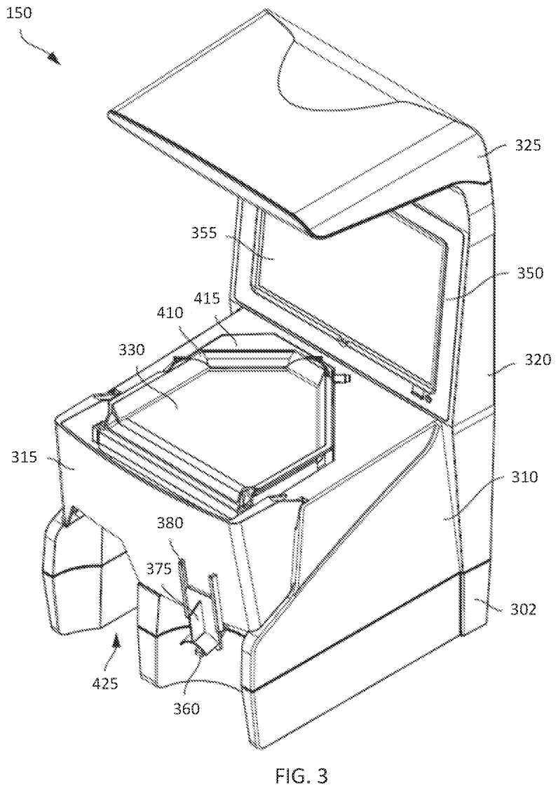

FIG. 3 illustrates one example of a pharmaceutical counting and packaging device 150. The pharmaceutical counting and packaging device 150 is a medication counting and packaging device for use in retail pharmacies that detects and counts a number of medications that are placed on the pharmaceutical counting and packaging device 150. The pharmaceutical counting and packaging device 150 displays the count and shares the count with other systems within the pharmacy. The pharmaceutical counting and packaging device 150 includes a base 305, a base portion 310, a front panel 315, a back panel 320, a top panel 325, and a tray 330. The top panel 325 includes a bar code sensor and a top camera. The bar code sensor is used to read bar codes printed on bulk storage containers and packaging vials. The top camera captures images of the contents of the tray 330. The back panel 320 may be made of a single piece or may include one or more pieces attached together. The illustrated back panel 320 includes a device slot 350 to receive a display device 355. For example, the back panel 320 may include a bottom piece to support the base portion 310 and a top piece to support the display device 355. The display device 355 is, for example, a graphics display coupled to a computing device of the pharmaceutical counting and packaging device 150. In some embodiments, the display device 355 is a tablet computer that is removably placed in the device slot 350. The display device 355 may include a camera to capture images in front of the display device above the tray 330. In some embodiments, the display device 355 also includes a camera to capture images of, for example, the bulk storage containers, the packaging vials, and the like. The camera of the display device 355 can also be used as a biometric scanner to verify the identity of a person operating the pharmaceutical counting and packaging device 150.

The front panel 315 includes a spout 360 and a panel funnel 370. In some embodiments, the pharmaceutical counting and packaging device 150 is used to fill vials. In these embodiments, the vial may be placed at the spout 360 to be filled with the counted medications on the tray 330. When the medications on the tray 330 are counted, the tray 330 may be lifted towards the front panel 315 such that the medications on the tray 330 are directed to the panel funnel 370. The panel funnel 370 slants towards the spout 360 to direct medications in the panel funnel 370 into a vial placed at the spout 360. The front panel 315 is made of transparent or translucent plastic material such that a user may observe the flow of medications from the tray 330 to the vial and ensure that no medications are stuck in the front panel 315. Additionally, the front panel 315 is removably attached to the pharmaceutical counting and packaging device 150 such that the front panel 315 may be easily removed for cleaning and servicing. In some embodiments, an opening cover 375 is provided on the front panel 315. The opening cover 375 may be moved between a first position that closes the spout 360 such that any medications are stopped from falling out of the spout 360 and a second position that opens the spout 360 such that the medications can flow out of the spout 360. The opening cover 375 moves between the first position and the second position using a sliding mechanism 380 such that the opening cover 375 can slide from the first position to the second position when an upward force is applied on the opening cover 375. The opening cover 375 returns to and rests in the first position when no upward force is applied on the opening cover 375.

The tray 330 is positioned above the base portion 310 and between the front panel 315 and the back panel 320. The tray 330 may be made of opaque or translucent plastic material In some embodiments, the tray 330 is made of translucent plastic material and includes a set of lights below the tray 330 such that the contents of the tray 330 may be illuminated from below the tray 330. In some embodiments the tray 330 may be made of transparent material with an opaque or translucent diffuser provided below the tray 330 in the base portion 310. The diffuser diffuses the light provided below the diffuser to provide a more even light distribution for the tray 330. The tray 330 rests above the rubber stoppers provided on the base portion 310 such that the tray 330 is provided above the diffuser.

The base portion 310 houses the electrical components of the pharmaceutical counting and packaging device 150. In some embodiments, the electrical components may be distributed between the base portion 310, the back panel 320, and the top panel 325. The base portion 310 supports the tray 330, for example over a diffuser. The base portion 310 includes a tray tilter 410, a slot cover 415, a slot funnel below the slot cover 415, and a packaging slot 425 below the slot funnel. The electrical components may be connected to rest of the pharmaceutical counting and packaging device 150 using a standard USB cable. An example pharmaceutical counting and packaging device 150 is described in U.S. Provisional Patent Application Ser. No. 62/984,143 titled “PHARMACEUTICAL COUNTING AND PACKAGING DEVICE,” the entire contents of which are hereby incorporated by reference herein.

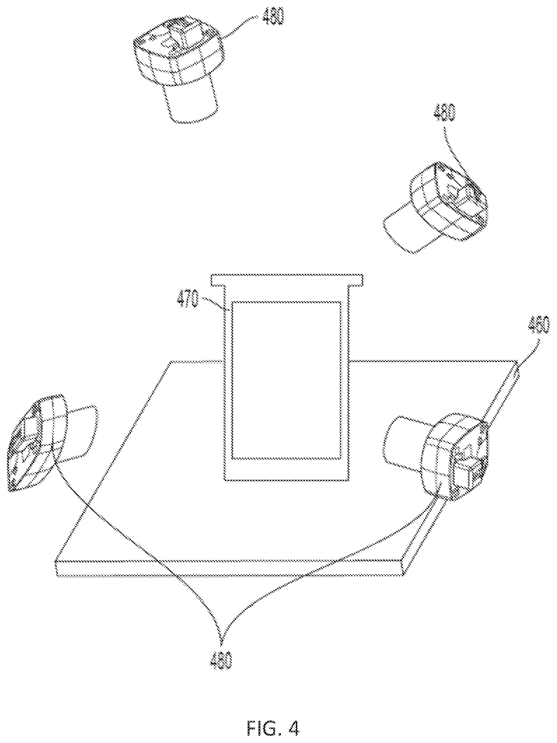

FIG. 4 illustrates the packaging slot 425 according to one example embodiment. The packaging slot 425 facilitates packaging of the counted medications into packages and verification of packaged medication. As illustrated in FIG. 4, the packaging slot 425 includes a platform 460, a package 470, and a verification system 480.

The platform 460 provides a base for receiving the package 470. In the example illustrated, the package 470 is a pill vial. In other embodiments, the package 470 may be another suitable container. The platform 460 may include additional features to help in the placement of the package 470 on the platform 460. For example, the platform 460 may include raised features to guide the placement of the package 470 in the packaging slot 425. In the example illustrated in FIG. 4, the verification system 480 is a verification camera system 480. The verification camera system 480 includes one or more cameras placed in the packaging slot 425 to capture the exterior and/or interior of the package 470. In the example illustrated, the packaging slot 425 includes three cameras placed 120 degrees apart to capture the exterior of the package 470. The cameras may be placed at the same height of the package 470 (for example, a vial) to capture a barcode or label placed on the package 470. The packaging slot 425 may also include a camera placed above the package to capture contents of the package 470. A lighting system may be used to illuminate the package 470 and the contents of the package 470 during image capture by the verification camera system 480. In some embodiments, the verification camera system 480 is used to verify that a correct packaging vial is placed on the platform 460. The verification camera system 480 programmatically unpeels the label to present the label information in a readable format. A counter electronic processor 510 then verifies the information from the unpeeled label to determine whether the correct packaging vial is placed on the platform 460.

In some embodiments, rather than multiple cameras, the verification camera system 480 may include one or more cameras provided in combination with mirror placed such that the one or more cameras can capture the entire label of the packaging vial. In some embodiments, the pharmaceutical counting and packaging device 150 may include a mechanism to rotate the platform 460. The one or more cameras may be used to capture multiple images of the packaging vial while the platform 460 is rotated to present several views of the packaging vial to the one or more cameras. In some embodiments, rather than moving the platform 460, the one or more cameras and/or the mirrors may be rotated around the platform 460 to capture several views of the packaging vial.

In some embodiments, the verification system 480 may include other verification components rather than a verification camera system 480. For example, the verification system 480 includes an RFID detector that detects an RFID chip integrated into the label of the packaging vial. The verification system 480 may include a barcode or QR code scanner to scan a 1-dimensional or 2-dimensional barcode provided on the packaging vial. In other embodiments, the packaging vial may be verified at other locations. For example, the packaging vial is verified at a remote pharmacist device 180, using a counter camera system 540, or at a separate verification machine or camera system. In some embodiments, a label application component may be provided in the pharmaceutical counting and packaging device 150 to apply a correct label to the packaging vial while the packaging vial is in the packaging slot 425 before or after the packaging vial is filled.

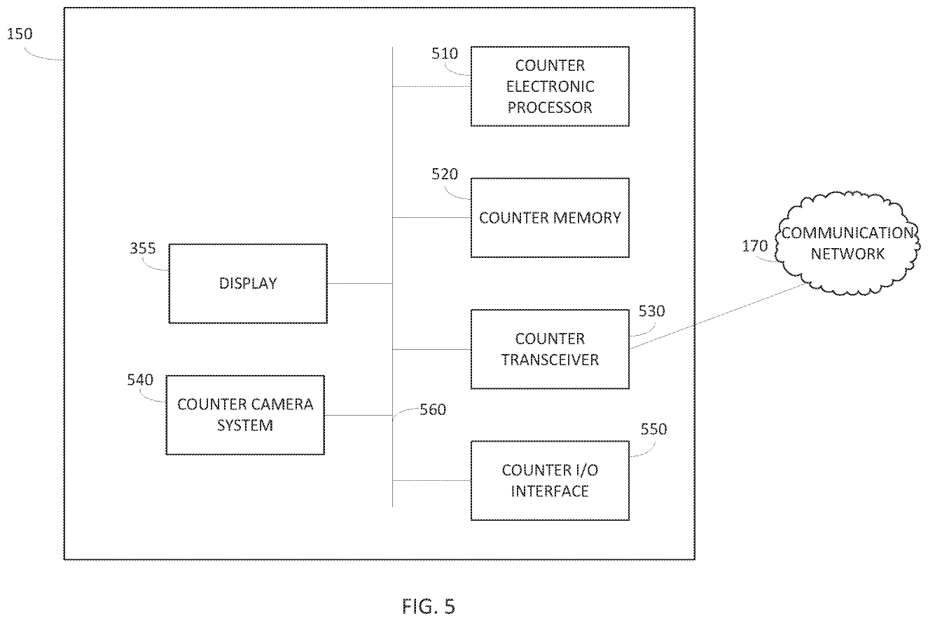

FIG. 5 schematically illustrates the pharmaceutical counting and packaging device 150. In the example illustrated, the pharmaceutical counting and packaging device 150 includes a counter electronic processor 510, a counter memory 520, a counter transceiver 530, a counter camera system 540, the display 355, and a counter input/output interface 550. The counter electronic processor 510, the counter memory 520, the counter transceiver 530, the counter camera system 540, the display 355, and the counter input/output interface 550 communicate over one or more control and/or data buses (e.g., a communication bus 560). FIGS. 3-5 illustrate only one exemplary embodiment of the pharmaceutical counting and packaging device 150. The pharmaceutical counting and packaging device 150 may include more or fewer components and may perform functions other than those explicitly described herein.

The counter electronic processor 510 and the counter memory 520 may be implemented similar to the electronic processor 110 and the device memory 120 as described above. Although certain drawings illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links. Here, for example, the counter electronic processor 510 may not be provided or may be provided in a different location and the functionality described below may be performed by the other electronic processors. In some embodiments, one or more of the electronic processors are provided in a cloud computer cluster arrangement, one or more of which may be executing none, all, or a portion of the applications of the pharmacy management system 100, sequentially or in parallel across the one or more electronic processors. One or more proxy servers or load balancing server may control which one or more electronic processors perform any part or all applications described herein.

The counter transceiver 530 enables communication from the pharmaceutical counting and packaging device 150 to the communication network 170. In some embodiments, the counter transceiver 530 may include separate transmitting and receiving components, for example, a transmitter and a receiver. The pharmaceutical counting and packaging device 150 through the communication network 170 may communicate with the pharmacy device 155, the remote pharmacist device 180 and databases, for example, the pharmacy database 190.

The counter input/output interface 550 may include one or more input mechanisms (e.g., a touch screen, a keypad, a button, a knob, and the like), one or more output mechanisms (e.g., a display, a printer, a speaker, and the like), or a combination thereof. The counter input/output interface 550 receives input from the input devices actuated by a user, and provides output to the output devices with which a user interacts. In some embodiments, as an alternative or in addition to managing inputs and outputs through the counter input/output interface 550, the pharmaceutical counting and packaging device 150 may receive user inputs, provide user outputs, or both by communicating with an external device, such as the device 355 or a console computer, over a wired or wireless connection.

The counter camera system 540 includes the top camera, the barcode sensor, the camera of the display device 355, and the verification camera system 480. The counter electronic processor 510 controls the camera system 540 to identify and count the medications on the tray 330. The counter electronic processor 510 controls the camera system 540 (that is, the barcode sensor) to verify that a correct bulk storage container is used for the prescription currently being filled. Additionally, the counter electronic processor 510 may also control the camera system 540 (that is, the barcode sensor) to verify that a correct package is being packaged by the pharmaceutical counting and packaging device 150. In some embodiments, the barcode scanner is a two-dimensional color camera which may also be used to capture images of the pills on the tray 330. The barcode scanner can be used in conjunction with the top camera to generate stereoscopic three-dimensional information of the tray 330 to obtain three-dimensional information of the medications on the tray 330. Other three-dimensional imaging systems, for example, laser based imaging systems, visual light based imaging system, and the like may be used to generate three-dimensional representations of the medications under inspection. The counter electronic processor 510 controls the counter camera system 540 to capture images of packaged pharmaceuticals as described above with respect to the verification camera system 480.

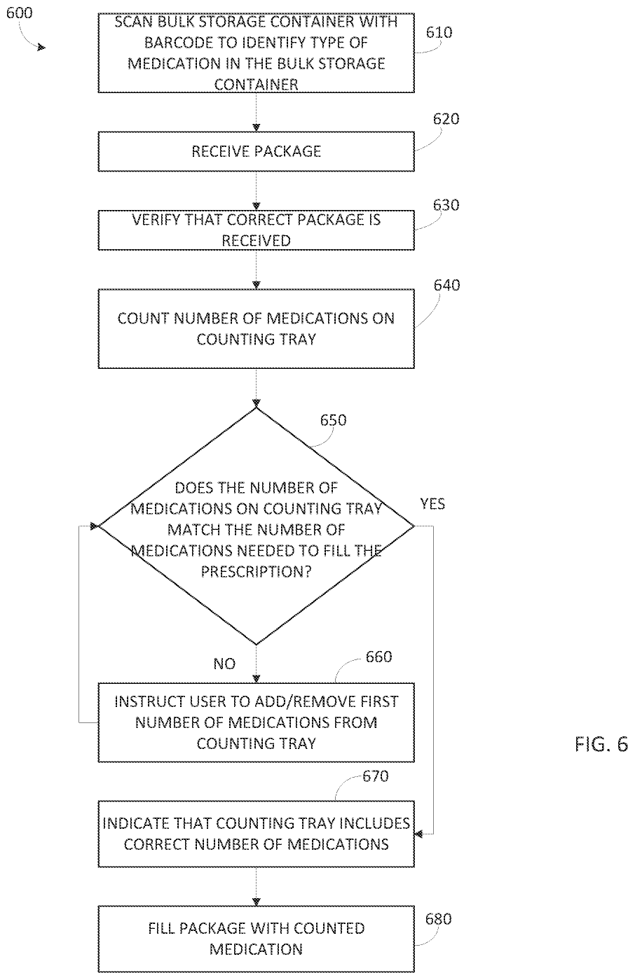

FIG. 6 depicts a method 600 of packaging medications for filling a prescription. Although the method 600 includes specific blocks, all the blocks need not be performed or performed in the order presented. When a technician receives a prescription from a customer, the pharmacy management system 100 generates an information sheet including a list of all the medications required to fill the prescription. The technician uses the information sheet to retrieve bulk containers including the medications in the information sheet. For example, the pharmacist retrieves the bulk container from storage shelves in the pharmacy. In some embodiments, the prescription is also provided to the pharmaceutical counting and packaging device 150. Specifically, the electronic processor 510 receives the prescription to be filled by the pharmaceutical counting and packaging device 150. An electronic copy of prescription may be transmitted to the pharmaceutical counting and packaging device 150 from the pharmacy management system 100. For example, a scanned copy of the prescription or prescription information entered by the technical are transmitted to the pharmaceutical counting and packaging device 150.

At block 610, the method 600 includes scanning, using the counter electronic processor 510 with the barcode scanner, a bulk storage container with a barcode to identify the type of medication in the bulk storage container. Once the pharmacist retrieves the bulk storage containers, the pharmacist scans the bulk storage containers at the pharmaceutical counting and packaging device 150. The barcode scanner scans the barcode on the bulk storage containers and provides the identification information read from the barcode to the counter electronic processor 510. The counter electronic processor 510 retrieves information pertaining to the medication within the bulk storage container using the identification information. For example, the counter electronic processor 510 communicates with the pharmacy database 190 to determine the type, the weight, the size, the color, etc. of the medication within the bulk storage container. Scanning the bulk storage containers at the pharmaceutical counting and packaging device 150 also verifies that the correct bulk storage containers were retrieved. Other types of scanning may also be used. For example, the camera system 540 (for example, a scanner) may be used to capture an image of the label of the bulk storage container. Optical character recognition may be applied to the captured image to determine the type of medication within the bulk storage container based on the label of the container. In some embodiments, the method 600 also includes capturing one or more images of the bulk storage container(s). The images of the bulk storage containers may be stored with the prescription information for each customer or patient.

At block 620, the method 600 includes receiving a package 470. The package 470 is, for example, a vial, a container, or the like in which the prescribed medications are packaged and provided to the customer. In some embodiments, the package 470 is received in the packaging slot 125. In some embodiments, the pharmaceutical counting and packaging device 150 may indicate to the user on the display 355 to place the package 470 in the packaging slot 425. In some embodiments, the package 470 may be received at other locations. For example, the package 470 may be received at a front packaging funnel of the pharmaceutical counting and packaging device 150.

At block 630, the method 600 includes verifying that the correct package 470 is received. The verification camera system 480 or other verification system as described above is used to verify that a correct label has been applied to the package 470. The counter electronic processor 510 captures images of the label of the package 470 using the verification camera system 480. For example, the verification camera system 480 includes three cameras placed 120 degrees apart in the packaging slot 425 to capture the label placed around a vial or container placed in the packaging slot 425. The counter electronic processor 510 activates the cameras to capture the images and combine the images to produce a single label image. The counter electronic processor 510 may use known image processing techniques to process the single label image to determine the information printed on the label placed on the package 470. The counter electronic processor 510 may then verify the information on the label with the information from the prescription to determine whether the correct package 470, for example, with the correct label is received in the packaging slot 450. The counter electronic processor 510 may also display the image or images of the label and the information on the label on the display 355 for verification by the pharmacist. Certain kinds of medications may require special packaging material. The counter electronic processor 510 may also use the images captured using the verification camera system 480 to determine whether the kind of package 470 used corresponds to the medications being filled in the package 470.

When the counter electronic processor 510 determines that the label on the package 470 is incorrect, corresponds to a different medication, is the wrong kind of package 470, or the like, the pharmaceutical counting and packaging device 150 may indicate to the user on the display 355 to replace the package 470 with a corrected package 470.

At block 640, the method 600 includes counting, using the counter electronic processor 510 with the counter camera system 540, the number of medications on the counting tray 330 of the pharmaceutical counting and packaging device 150. After scanning a bulk storage container, the pharmacist pours medications from the bulk storage container onto the counting tray 330. The counter electronic processor 510 captures an image of the counting tray 330 using the counting camera system 540 to determine the number of medications on the counting tray 330. The counter electronic processor 510 processes the image captured by the counter camera system 540 using known image processing systems to determine the number of medications on the counting tray 330. Particularly, the counter electronic processor 510 analyzes the shadows cast by the medications to determine the number of medications on the counting tray 330. In some embodiments, the pharmaceutical counting and packaging device 150 continuously captures the image of the counting tray 330 at regular time intervals (e.g., every 0.5 seconds) to update the count of the medications.

The counter electronic processor 510 also captures the visible light images of the medications on the counting tray 330. The counter electronic processor 510 processes the IR images and the visible light images to isolate portions of the images corresponding to individual medications. The counter electronic processor 510 determines an expected medication type to be counted using the pharmaceutical counting and packaging device based on the scanned bulk storage container. The counter electronic processor 510 compares the isolated images of the medications on the counting tray with the images of expected medication types pre-stored in the counter memory 520 or received from the pharmacy database 190. When the medications on the counting tray 330 do not match the expected medications type, the counter electronic processor 510 prompts the user to remove medications from the counting tray 330. For example, the counter electronic processor 510 provides an indication on the display 430 that the medications on the counting tray do not match the expected medications.

At block 650, the method 600 determines whether the number of medications on the counting tray 330 matches the number of medications needed to fill the prescription. The pharmaceutical counting and packaging device 150 may receive the prescription information or the label sheet information from the pharmacy management system 100. The pharmaceutical counting and packaging device 150 determines the number of medications needed to fill the prescription from the prescription information or the label sheet information. Particularly, the technician may scan the information sheet generated by the pharmacy management system 100 using the barcode scanner. The pharmacy management system 100 sends the information included on the information sheet to the pharmaceutical counting and packaging device 150. The electronic processor 110 of the pharmacy management system 100 communicates with the pharmaceutical counting and packaging device 150 using the transceiver 130 and the communication network 170 to provide the information on the information sheet to the counter electronic processor 510. The counter electronic processor 510 retrieves the information received from the pharmacy management system 100 based on scanning the bar code on the information sheet. The pharmaceutical counting and packaging device 150 compares the number of medications on the counting tray 330 to the number of medications needed to fill the prescription.

When the number of medications on the counting tray 330 does not match the number of medications needed to fill the prescription, the pharmaceutical counting and packaging device 150 instructs the user to add/remove a first number of medications from the counting tray 330, at block 660. The pharmaceutical counting and packaging device 150 determines the difference between the number of medications on the counting tray 330 and the number of medications needed to fill the prescription and indicates to the user (e.g., using the display 355) to add or remove the first number of medications corresponding to the difference between the number of medications on the counting tray 330 and the number of medications needed to fill the prescription. For example, if 30 medications are needed to fill the prescription, but 32 medications are poured onto the counting tray 330, the pharmaceutical counting and packaging device 150 instructs the user to remove 2 pills and return the pills to the bulk container. Conversely, if 30 medications are needed to fill the prescription, but 28 medications are poured onto the counting tray 330, the pharmaceutical counting and packaging device 150 instructs the user to add 2 pills to the counting tray 330 from the bulk container. As described above, the method 600 then returns to block 650 to continuously determine the number of medications on the counting tray 330.

When the number of medications on the counting tray 330 matches the number of medications needed to fill the prescription, the pharmaceutical counting and packaging device 150 indicates that the counting tray 330 includes the correct number of medications, at block 670. The pharmaceutical counting and packaging device 150 may also process the images captured to identify whether any medications are broken or whether the counting tray 330 includes any debris. The pharmaceutical counting and packaging device 150 instructs the technician to remove the broken medications or debris from the counting tray 330. In some embodiments, when the number of medication on the counting tray 330 matches the number of medications needed to fill the prescription, the method 600 also includes capturing, using the counter camera system 540, an image of the pills on the counting tray.

At block 680, the method 600 includes filling the package 470 with the counted medication. The counter electronic processor 510 deactivates the lockout mechanism of the counting tray 330 such that the user may lift the counting tray 330 to fill the medications in the package 470 received in the packaging slot 425. The pharmacist then empties the medications on the counting tray 330 into the package 470. Once the package 470 is filled, the method 600 may also including capturing, using the counter camera system 540, an image of pills in the package 470. For example, an instruction may be displayed on display device 355 asking the technician to place the filled package 470 (for example, an unclosed pill vial) on the counting tray 330. The counter camera system 540 captures an image from the top of the package 470 including the pills within the package 470. The method 600 is repeated for each medication in the prescription to fill different packages 470. This allows the pharmacist to fill the exact number of medications needed to fill the prescription into the packages 470 and does not need to dump excess medications from the packages 470 back into the bulk storage container after packaging.

The pharmaceutical counting and packaging device 150 may automatically apply a label to the packages after each job is completed or may include a printer to print a label that is then applied by the user to the filled packages. In some embodiments, the pharmaceutical counting and packaging device 150 may include a mechanism to automatically apply a cap or lid to the package being filled by the pharmaceutical counting and packaging device 150.

In addition to counting the number of medications, the pharmaceutical counting and packaging device 150 may also be used to determine one or more characteristics of the medications. For example, the pharmaceutical counting and packaging device 150 may be used to determine the color and dimensions of the medications. The NDC information received from the pharmacy database 190 generally includes information regarding the color and size of the medications within set ranges. The pharmacy management system 100 communicates the color and size information with the pharmaceutical counting and packaging device 150 for verification during packaging. However, there may be variations in the shade of the color (e.g., shade of blue) and size during manufacturing. For example, when manufacturing the medications, the medications do not always come out with the same color quality. A medication may be identified as blue in the database, but the shade of blue may be slightly different in the actual medication. Similarly, a medication may be identified in the database as having a diameter of 2 to 3 millimeters, and the actual size of the medication may be anywhere within that range.

In some embodiments, the pharmaceutical counting and packaging device 150 determines whether the type of medication on the counting tray matches the type of medication of the bulk storage container and/or the type of medication listed on the prescription. Specifically, the electronic processor 510 compares the characteristics of the medication on the counting tray with known characteristics of an expected medication to determine whether the correct type of medications are received on the counting tray. The pharmaceutical counting and packaging device 150 may provide this verification information as indications to the remote pharmacist. The pharmaceutical counting device 150 may transmit a first indication when the type of medication on the counting tray corresponds to the type of medication of the bulk storage container and/or the type of medication listed on the prescription and may transmit a second indication when the type of medication on the counting tray does not correspond to the type of medication of the bulk storage container and/or the type of medication listed on the prescription. For example, the electronic processor 510 may alter the image of the pills on the counting tray to provide a green border (e.g., a first indication) around each pill verified to correspond to an expected pill and provide a red border (e.g., a second indication) around each pill that does not correspond to an expected pill.

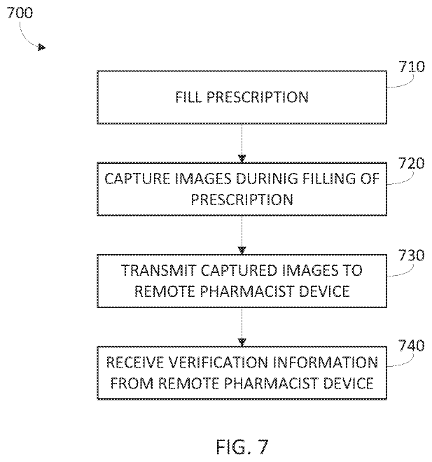

FIG. 7 depicts a method 700 for remote verification for the pharmaceutical counting and packaging device 150. Although the method 700 includes specific blocks, all the blocks need not be performed or performed in the order presented. At block 710, the method 700 includes filling, using the pharmaceutical counting and packaging device 150, a prescription. An example method 600 for filling a prescription is described above. At block 720, the method 700 includes capturing, using the pharmaceutical counting and packaging device 150, images during filling of the prescription. As discussed above in method 600, the pharmaceutical counting and packaging device 150 captures images of the pills on the counting tray, images of the label applied to the package 470, and images of the pills in the package 470.

At block 730, the method 700 includes transmitting, using the pharmaceutical counting and packaging device 150, the captured images to the remote pharmacist device 180. In one example, the prescription information and the captured images are transmitted directly to the remote pharmacist device 180 over the communication network 170. In another example, the prescription information and the captured images are uploaded to the pharmacy database 190. the remote pharmacist device 180 then downloads the prescription information from the pharmacy database 190. Specifically, the image of the bulk storage container, the image of the label applied to the package, the image of the pills on the counting tray, and the image of the pills in the package are transmitted. In some embodiments, the prescription or information pertaining to the prescription may also be transmitted to the remote pharmacist for verification.

As noted above, the remote pharmacist device 180 may be located at another location that is remote from where the prescription is being filled. For example, a first pharmacist at pharmacy A may have an abundance of prescriptions to fill, while a second pharmacist at pharmacy B may currently be light on work. The pharmacies A and B may already share an existing pharmacy management system, such as a database of customers, histories, and prescription orders. In order to help the first pharmacist at pharmacy A, the second pharmacist at pharmacist at pharmacy B may log onto the existing pharmacy management system to check a queue of prescriptions that need filling at pharmacy A. The second pharmacist may select one of the prescriptions to be filled from the queue to obtain information about the prescription order (e.g., customer, type of pharmaceutical, number of pharmaceuticals, etc.). Using this information, however, the pharmacist is unable to determine what pharmaceutical are actually packaged for delivery to the customer.

The second pharmacist at the pharmacy B, therefore, may use the information received from the pharmaceutical counting and packaging device 150 to supplement his or her review of the prescription order. For example, the prescription information and captured images from the pharmaceutical counting and packaging device 150 may be displayed to the second pharmacist at pharmacy B. In some embodiments, the prescription information and captured images may be displayed on a separate display (e.g., laptop computer, tablet computer, monitor, etc.) from the existing pharmacy management system. In other embodiments, the prescription information and captured images may be displayed on the same display used by the existing pharmacy management system via, for example, a split screen. The prescription information and the captured images are displayed on the display of the remote pharmacist device 180 for verification by the remote pharmacist. For example, the prescription images, the label images, and images of pills in the package 470 may be displayed on a grid arranged in rows and columns. The different information and images may be distributed along the rows, while the different packages 470 are distributed along the columns. For example, a first row may display label images and a second row may display images of pills in the package 470. A first column may display information and images related to a first package 470 and a second row may display information and images related to a second package 470. In some embodiments, one or more images of expected pills may also be displayed to aid in verification.

The second pharmacist then compares the prescription order from the existing pharmacy management system to the information obtained from the pharmaceutical counting and packaging device to determine whether the prescription order was properly filled and packaged at the pharmaceutical counting and packaging device 150 in pharmacy A.

At block 740, the method 700 includes receiving, at the pharmaceutical counting and packaging device 150, verification information from the remote pharmacist device 180. The verification information may be provided through the existing pharmacy management system. The display of the existing pharmacy management system may provide options for the remote pharmacist to accept the packaging information or to reject and provide correction information to the technician.

Once the remote pharmacist verifies the packaging information, the verification information is provided to the pharmaceutical counting and packaging device 150. The verification information includes, for example, whether the packaging information was accepted by the remote pharmacist indicating that the packages 470 are correctly packaged or correction information to correct any incorrectly packaged packages 470. The verification information may be provided on the display 355 for the technician. The technician may then correct any incorrectly packaged packages 470 and return the repackaged packages to the remote pharmacist or provide the packages to the customer.

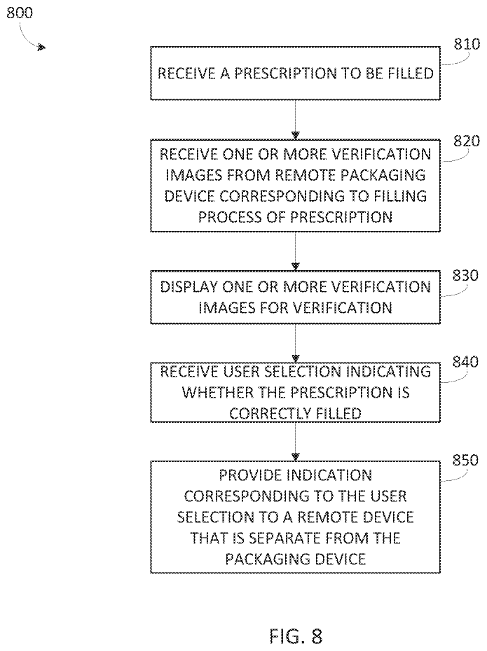

FIG. 8 depicts a method 800 for remote verification performed, for example, at the remote pharmacist device 180. Although the method 800 includes specific blocks, all the blocks need not be performed or performed in the order presented. At block 810, the method 800 includes receiving, using the device electronic processor 210, a prescription to be filled. As discussed above, an organization (e.g., pharmacy chain, hospital network, and the like) may have several linked pharmacies including a central database 190 that stores all prescription and filling information. The prescription may be physically or electronically received at any one of the network pharmacies. When the prescription is physically received, a technician may manually enter the details of the prescription or scan the prescription. The prescription is then stored in the database 190. When the remote pharmacist is ready to verify a prescription being filled at a different location, the remote pharmacist retrieves the prescription from the database 190 at the remote pharmacist device 180.

At block 820, the method 800 includes receiving, using the device electronic processor 210, one or more verification images from a remote packaging device corresponding to the filling process of prescription. The remote packaging device is, for example, the pharmaceutical counting and packaging device. As discussed above in method 600, the pharmaceutical counting and packaging device 150 captures images of bulk storage containers, images of the pills on the counting tray, images of the label applied to the package 470, and images of the pills in the package 470. These images are captured during a filling process of the prescription. The images are stored in the database 190 correspond to the prescription being filled. The device electronic processor 210 retrieves the one or more image associated with the filling process corresponding to the prescription from the database 190.

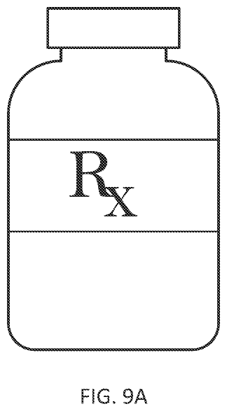

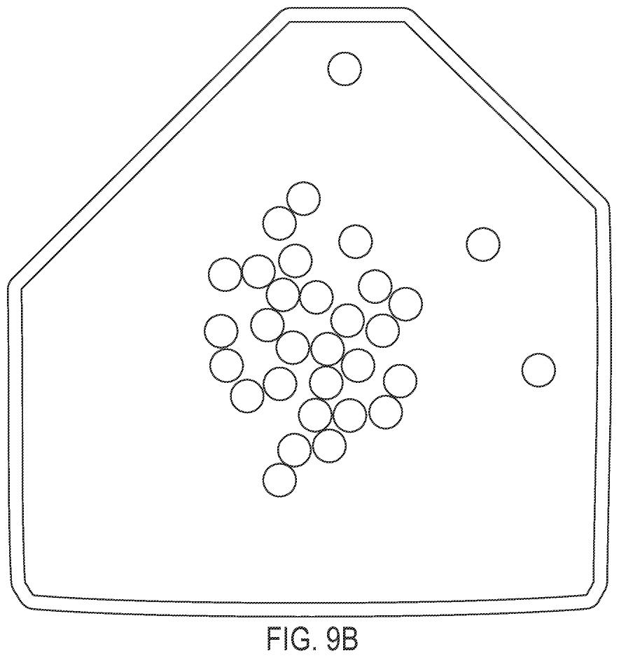

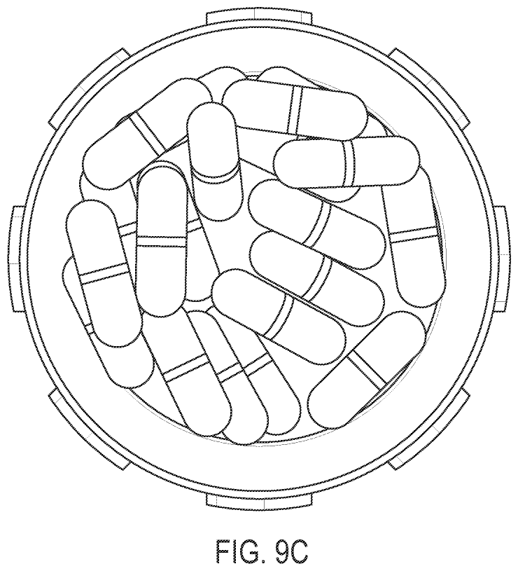

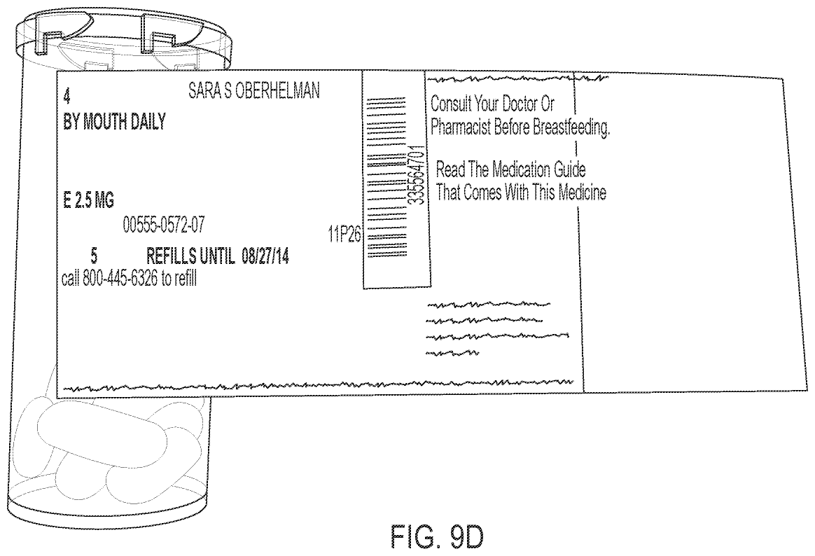

FIGS. 9A-9D illustrate example images captured by the pharmaceutical counting and packaging machine. FIG. 9A illustrates an image of a bulk storage container 910 captured by the

At block 830, the method 800 includes displaying, on a local user interface, the one or more verification images for verification. The device electronic processor 210 displays the one or more verification images on the device input/output interface 240 (e.g., the local user interface) for verification by the remote pharmacist (or local pharmacist from the perspective of the remote pharmacist device 180). The one or more images may be displayed in conjunction with the prescription or information pertaining to the prescription.

At block 840, the method 800 includes receiving, using the local user interface, a user selection indicating whether the prescription is correctly filled. The display screen displaying the verification images may provide options to accept or rejection the filled package based on the images. The user may accept, rejection, or request changes using the device input/output interface 240.

At block 850, the method 800 includes providing, using the device electronic processor 210, an indication corresponding to the user selection to a remote device that is separate from the packaging device. The device electronic processor 210 provides the user selection to the database 190, which can then be accessed by the pharmacy device 155 at a pharmacy that is filling and dispensing the prescription to the customer. For example, an alert may be provided on the pharmacy device 155 indicating that verification information for the prescription is received. The technician may then retrieve the verification information from the database 190 and take corresponding action. For example, the technician may sell the package 470 including the prescription medication to the customer, make the changes the requested by the remote pharmacist, or refill the prescription.

One advantage of the above methods is that volume of work may be evenly distributed among multiple locations of a retail pharmacy.

1-4. (canceled)

5. A pharmaceutical counting and packaging device comprising:
a counting tray configured to receive medications for counting;
a camera system for capturing images;
a transceiver; and
an electronic processor coupled to the camera system and the transceiver and configured to
receive a prescription to be filled by the pharmaceutical counting and packaging device;
capture, using the camera system, an image of a label applied to a package receiving a first type of medication listed on the prescription;
count a number of medications on the counting tray;
determine whether the number of medications on the counting tray matches the number of medications needed to fill the prescription;
in response to the number of medications on the counting tray matching the number of medications needed to fill the prescription
capture, using the camera system, an image of medications on the counting tray, and
capture, using the camera system, an image of medications in the package; and
transmit, using the transceiver, the image of the label applied to the package, the image of medications on the counting tray, and the image of medications in the package to a remote device.

6. The pharmaceutical counting and packaging device of claim 5, wherein the electronic processor is further configured to
determine whether a second type of medication on the counting tray corresponds to a third type of medication listed on the prescription;
transmit a first indication to the remote device when the second type of medication on the counting tray corresponds to the third type of medication; and
transmit a second indication to the remote device when the second type of medication on the counting tray does not correspond to the third type of medication.

7. The pharmaceutical counting and packaging device of claim 5, wherein the electronic processor is further configured to transmit the prescription to the remote device.

8. The pharmaceutical counting and packaging device of claim 5, wherein the electronic processor is further configured to receive verification information corresponding to the prescription, wherein the verification information includes an indication of whether the package is correctly packaged by the pharmaceutical counting and packaging device.

9. (canceled)

10. A method for remote verification for a pharmaceutical counting and packaging device, the method comprising:
receiving a prescription to be filled by the pharmaceutical counting and packaging device;
capturing, using a camera system of the pharmaceutical counting and packaging device, an image of a label applied to a package receiving a first type of medication listed on the prescription;
counting, using an electronic processor of the pharmaceutical counting and packaging device, a number of medications on a counting tray of the pharmaceutical counting and packaging device;
determining, using the electronic processor, whether the number of medications on the counting tray matches the number of medications needed to fill the prescription;
in response to the number of medications on the counting tray matching the number of medications needed to fill the prescription
capturing, using the camera system, an image of medications on the counting tray, and
capturing, using the camera system, an image of medications in the package; and
transmitting, using a transceiver of the pharmaceutical counting and packaging device, the image of the label applied to the package, the image of medications on the counting tray, and the image of medications in the package to a remote device.

11. The method of claim 10, further comprising:
determining whether a second type of medication on the counting tray correspond to one or both of the first type of medication and a third type of medication listed on the prescription;
transmitting a first indication to the remote device when the second type of medication on the counting tray correspond to one or both of the first type of medication and the third type of medication; and
transmitting a second indication to the remote device when the second type of medication on the counting tray does not correspond to one or both of the first type of medication and the third type of medication.

12. The method of claim 10, further comprising transmitting the prescription to the remote device.

13. The method of claim 10, further comprising receiving verification information corresponding to the prescription, wherein the verification information includes an indication of whether the package is correctly packaged by the pharmaceutical counting and packaging device.

14. (canceled)

15. A method for remote verification comprising:
receiving, at an electronic processor, a prescription to be filled;
receiving, using the electronic processor, one or more verification images from a remote packaging device corresponding to a filling process of the prescription;
displaying, on a local user interface, the one or more verification images for verification;
receiving, using the local user interface, a user selection indicating whether the prescription is correctly filled; and
providing, using the electronic processor, an indication corresponding to the user selection to a remote device that is separate from the remote packaging device.

16. The method of claim 15, wherein the verification images include an image of a label applied to a package including the medications listed on the prescription and an image of medications in the package

17. The method of claim 16, wherein the verification images further include an image of medications on a counting tray of the packaging device.
